(19) United States
(12) Patent Application Publication
(10) Pub. No.: US 2026/0334923 A1
(43) Pub. Date: Oct. 29, 2026

(54) DEVICE AND METHOD FOR PREDICTING ABILITY TO IMPLANT IN UTERUS AND ESTIMATING DELIVERY TIMING

(71) Applicant: THE UNIVERSITY OF OSAKA, Osaka (JP)

(72) Inventors: Hitomi NAKAMURA, Osaka (JP); Tadashi KIMURA, Osaka (JP); Takayoshi HOSONO, Osaka (JP)

(21) Appl. No.: 19/486,257

(22) PCT Filed: May 23, 2024
(86) PCT No.: PCT/JP2024/019026

§ 371 Date: Nov. 20, 2025

(30) Foreign Application Priority Data
May 23, 2023 (JP) 2023-084900

(51) Int. Cl. A61B 10/00; A61B 5/00; A61B 5/0538

(52) U.S. Cl. CPC A61B 10/0012; A61B 5/0538; A61B 5/6875; A61B 5/7278; A61B 2010/0016

(57) ABSTRACT

The present invention provides a device and a method for predicting the ability to implant in the uterus of a mother, and a device and a method for estimating delivery timing. This device for predicting the ability to implant in the uterus of a subject comprises: an electrode that is inserted up to a position which is either in the uterine cavity or intravaginal cavity of the subject; a measurement means for measuring the bioelectrical impedance at the position; and a predicting means for predicting the ability to implant, using the measured bioelectrical impedance value as an index. A device for estimating the delivery timing of the subject according to the present invention comprises: an electrode that is brought into contact with the mucosal epithelium at a position in the uterine cavity or intravaginal cavity of the subject; a measuring means of measuring the bioelectric impedance generated between the electrode and the mucosal epithelium; and an estimating means of estimating the delivery timing of the subject using the measured value of the bioelectric impedance as an index.

### TECHNICAL FIELD

The present invention relates to a device and a method for predicting the maternal receptivity for implanting a fertilized egg in the uterus (uterine receptivity), and a device and method for estimating the delivery timing.

### BACKGROUND ART

Human infertility is defined as the failure to conceive after 12 months or more of regular unprotected sexual intercourse. It is estimated that 10 to 15 percent of all couples of reproductive age suffer from infertility. In recent years, infertility is thought to be on the rise due to factors such as later marriage and later childbirth in accordance with women entering the workforce, as well as the spread of sexually transmitted diseases.

The major causes of female infertility are disorders of ovaries (ovulation) and tubal (transporting eggs) such as blocked fallopian tubes. However, it is sometimes not possible to explain the causes of infertility. Many cases of the unexplained infertility are thought to be due to implantation failure.

The “implantation” is the process where a fertilized egg (embryo) attaches to the lining of the uterus, beginning pregnancy. When there is some problem with the receiving endometrium during this implantation process and implantation does not occur as a result, this is called “implantation failure.” In other words, the “implantation failure” refers to a condition in which fertilization is successful and the embryo develops normally, but it moves to the uterine cavity, but implantation does not occur normally.

Based on the concept that human implantation is controlled by three factors: the hormonal environment, endometrial function, and morphology, (1) blood progesterone levels have been examined, (2) endometrial thickness has been measured by using an ultrasonic diagnostic device, and (3) endometrial tissue has been collected and examined histologically. The above-mentioned method (3) is not routinely performed in clinical practice because it is highly invasive and is not a prospective test, even though the endometrial implantation capacity changes with each menstrual cycle. The above methods (1) and (2) are still routinely used in clinical settings.

However, it has been reported that the above methods (1) and (2) are insufficient for prospectively evaluating the implantation capacity of the uterus. Furthermore, these methods routinely result in far too many cases of pregnancy not being achieved despite the fact that these parameters have no problem.

Assisted reproductive technologies (ART) treatment has long been one of the common options for infertility treatment, but the pregnancy rate is approximately 20 to 30% worldwide, although this varies greatly depending on the maternal age, so it cannot be said to be a sufficient efficiency of treatment.

The process leading to pregnancy can be broadly divided into (i) ovulation, (ii) fertilization, and (iii) implantation. However, in current infertility treatments, when there are any problems with the above (i) and (ii), there are diagnostic and treatment methods available, but the current situation is that there is never diagnostic method for the above (iii).

Further, in the assisted reproductive technologies (ART) treatment, 83% to 89% of embryos are able to develop outside the body, but do not result in pregnancy. One-third of this cause is thought to be caused by problems with the embryo itself, while the remaining two-thirds are thought to be caused by problems with the recipient uterus, known as implantation failure.

For these reasons, there is a need for diagnosis and treatment of the implantation failure in current infertility treatments. The low success rate of in vitro fertilization often results in excessive financial, emotional and physical strain on the couples struggling with infertility. Considering the perinatal prognosis, it is necessary for couples who wish to have children to become pregnant as soon as possible, and for that purpose, efficient infertility treatment is desirable.

That is, by prospectively evaluating the uterine implantation capacity for each menstrual cycle, it is possible to provide treatment that is appropriate for that menstrual cycle and is efficient, thereby reducing the physical, psychological, and economic burden on the patients.

Accordingly, although diagnosis and treatment of implantation failure are required in current infertility treatments, there is currently no practical procedure for prospectively evaluating the implantation capacity of the uterus, and no diagnostic procedure for implantation failure has been established.

In contrast, because vaginal mucosal impedance in rats is high during proestrus when not pregnant, this vaginal mucosal impedance has been put to practical use as a parameter indicating the breeding period (Non-Patent Document 1). Further, it has been reported that vaginal mucosal impedance is elevated during estrus in mice (Non-Patent Documents 2 and 3). However, there have been no attempts to evaluate the implantation capacity of the uterus by using this parameter.

Therefore, the inventors conducted extensive experiments by using a mouse model of human implantation failure, and from the results, developed a method and a device for measuring the implantation capacity of the endometrium of the subject by using at least one of the potential differences (e.g., oxidation-reduction potential (ORP)) in the uterine cavity, local impedance in the uterine cavity, and vaginal mucosal impedance as an index (Patent Document 1).

### CITATION LIST

### Patent Document

Patent Document 1: International Patent Publication No. WO2012/070569

### Non-Patent Document

Non-Patent Document 1: Aplin JD et al., Ann N Y Acad Sci. 2008 Apr; 1127:116-20.
Non-Patent Document 2: FEBS Lett. 2006, Vol. 580, pp. 2717-2722
Non-Patent Document 3: Masao Koto et al., “Determining the optimal mating time for F344 rats by using vaginal impedance analysis,” Experimental Animal Technology, 22(2), 82-85, 1987

### SUMMARY OF THE INVENTION

### Problem to be Solved by the Invention

The present inventors further conducted clinical studies of bioelectrical impedance measurements in humans and additional experiments of bioelectrical impedance measurements in mouse models, and have obtained novel findings regarding prediction of uterine implantation capacity and estimation of delivery timing (or prediction of preterm delivery).

Therefore, the object of the present invention is to provide a device and a method for predicting the implantation capacity of the uterus of mother. Furthermore, another object of the present invention is to provide a device and a method for estimating the delivery timing.

### Means to Solve the Problems

The present invention has been completed based on the above findings and includes the following embodiments.

That is, a first embodiment of the present invention provides a device for predicting the implantation capacity of the uterus of a subject, which comprises:

an electrode that is inserted up to a position which is either in the uterine cavity or intravaginal cavity of the subject;

a measurement means for measuring the local bioelectrical impedance at the position; and

a predicting means for predicting the implantation capacity by using the measured local bioelectrical impedance as an index.

Further, in the device according to the first embodiment of the present invention, it is preferable that the predicting means includes:

a comparison means for comparing the measured value with at least one of a predetermined threshold value and a control value which is a value of local bioelectrical impedance at a position in the uterine cavity or vaginal cavity previously measured in a fertile woman; and

a determination means for determining that the implantation capacity in the corresponding menstrual cycle is insufficient when the measured value is higher than the corresponding threshold value or control value, and for determining that the implantation capacity in the corresponding menstrual cycle is good when the measured value is the same as or lower than the corresponding threshold value or control value.

Further, it is preferable that the device according to the first embodiment of the present invention further comprises an output means for outputting the predicted implantation capacity.

A second embodiment of the present invention provides a method, comprising the steps of:
inserting an electrode up to a position which is either in the uterine cavity or intravaginal cavity of a subject;
measuring the local bioelectrical impedance at the position; and
predicting the implantation capacity by using the measured local bioelectrical impedance as an index.

A third embodiment of the present invention provides a device for estimating the delivery timing of a subject (or predicting the possibility of premature delivery), comprising:
an electrode that is brought into contact with the mucosal epithelium at a position in either the uterine cavity or the vaginal cavity of the subject;
a measurement means that measures the bioelectrical impedance generated between the electrode and the mucosal epithelium; and
an estimation (or prediction) means that estimates the delivery timing of the subject (predicts the possibility of premature delivery) by using the measured value of the bioelectrical impedance as an index.

In the device according to the third embodiment of the present invention, it is preferable that the estimation (or prediction) means includes:
a comparison means for comparing the measured value with at least one of a predetermined threshold value and a control value which is a value of local bioelectrical impedance at a position in the uterine cavity or vaginal cavity previously measured in a fertile woman; and
a determination means for determining that the delivery timing of the subject is relatively early (or that there is a relatively high possibility of preterm delivery) when the measurement value is lower than the corresponding threshold value or control value, and for determining that the delivery timing of the subject is normal or relatively late (or that there is a relatively low possibility of preterm delivery) when the measurement value is the same as or higher than the corresponding threshold value or control value.

It is preferable that the device according to the third embodiment of the present invention further comprises an output means for outputting the estimated delivery timing.

A fourth embodiment of the present invention provides a method, comprising the steps of:
bring an electrode into contact with the mucosal epithelium at a position in either the uterine cavity or the vaginal cavity of a subject;
measuring the local bioelectrical impedance at the position; and
estimating the delivery timing of the subject (or predicting the possibility of preterm delivery) by using the measured value of the bioelectrical impedance as an index.

### EFFECT OF THE INVENTION

According to one embodiment of the present invention, a device and a method for predicting the implantation capacity of a uterus of a mother can be provided. That is, by prospectively evaluating the implantation capacity of the uterus, it is possible to provide efficient infertility treatment.

As a result, it is possible to have children sooner than currently possible, and the cost of infertility treatment required for pregnancy is reduced. Thereby, it is possible to enable couples who have not been able to receive infertility treatment for financial reasons to receive the treatment. This will ultimately lead to an increase in the birth rate, which is expected to help secure economic and social foundations in Japan, which are currently being shaken by the problem of the declining birthrate.

Further, by ensuring a high probability of success in infertility treatment, it is possible to reduce the number of ovarian stimulation and egg collection procedures, which are expensive and place a great burden on the mother. Furthermore, by improving the pregnancy rate per embryo transfer, it is expected that the number of transferred embryos will be limited to one, eliminating iatrogenic multiple pregnancies resulting from infertility treatment, which is currently a problem in perinatal medicine.

According to another embodiment of the present invention, it is possible to provide a device and a method for estimating the delivery timing (or predicting the possibility of preterm delivery). That is, the predictive parameters for preterm delivery have a high negative predictive rate. By using this in combination with known parameters, it is possible to select cases that require aggressive medical intervention, and provide the necessary treatment to those who need it.

### BRIEF DESCRIPTION OF THE DRAWINGS

FIG. 1 is a block diagram showing a schematic configuration of the device 1 according to the embodiment 1 of the present invention.

FIG. 2 is a block diagram showing a schematic configuration of the device 101 according to a modified example of the embodiment 1 of the present invention.

FIG. 3 is a schematic diagram of the measuring electrode 202 that can be used in the embodiment 2 of the present invention.

FIG. 4 is a graph showing the measured results of the bioelectrical impedance measured in the uterine cavity at three different times in Experimental Example 1.

FIG. 5 shows, with respect to the bioelectrical impedance values measured at the local position in the uterine cavity in Experimental Example 1, (A) a diagram of the results of comparing between two groups, one that ultimately resulted in pregnancy and the other that did not, and (B) a diagram of the ROC curve (Receiver Operating Characteristic curve) and the AUC curve (Area Under the ROC curve) for whether the measured bioelectrical impedance values measured at the local position in the uterine cavity can predict whether pregnancy will not occur in this menstrual cycle.

FIG. 6 shows, with respect to the bioelectrical impedance measured at the local position in the vaginal in Experimental Example 2, (A) a diagram of the results of comparison between mice in which preterm delivery was induced by administration of Mifepristone and control mice, and (B) a diagram of the results of ROC analysis.

FIG. 7 shows, with respect to the bioelectrical impedance measured at the local position in the vaginal in Experimental Example 2, (A) a diagram of the results of comparison between mice in which preterm delivery was induced by administration of LPS and control mice, and (B) a diagram of the results of ROC analysis.

FIG. 8 shows, with respect to the bioelectrical impedance of the vaginal region measured by using frequencies of 5 kHz, 10 kHz, 50 kHz, 125 kHz, or 250 kHz in Experimental Example 2-3, a diagram of the results of comparing between mice in which preterm delivery was induced by administration of Mifepristone and control mice.

FIG. 9 is a diagram showing the results of roc analysis of the measurement results of FIG. 8.

FIG. 10 shows, with respect to the bioelectrical impedance of the vaginal region measured by using frequencies of 5 kHz, 10 kHz, 50 kHz, 125 kHz, or 250 kHz in Experimental Example 2-4, a diagram of the results of comparing between mice in which preterm delivery was induced by administration of LPS and control mice.

FIG. 11 is a diagram showing the results of ROC analysis of the measurement results of FIG. 10.

FIG. 12 shows, with respect to the mice in which preterm delivery was induced by administration of LPS in Experimental Examples 2-4, a diagram of the relationship between the successive frequencies and AUC.

### MODE FOR CARRYING OUT THE INVENTION

Hereinafter, an apparatus according to a representative embodiment of the present invention will be described in detail with reference to the drawings, but the present invention is not limited thereto. However, since the drawings are intended to conceptually explain the present invention, there are the cases where the dimensions, ratios, or numbers may be exaggerated or simplified in order to facilitate understanding.

### 1. Embodiment 1

Embodiment 1 is described as one embodiment of the present invention. Embodiment 1 relates to the prediction of the implantation capacity of the uterus.

### (1) Method for Predicting Uterine Implantation Capacity

According to the method for predicting the uterine implantation capacity of the present embodiment, the bioelectrical impedance in the uterine cavity of the subject is used as an indicator. The subjects supposed here are women who wish to have children, particularly women undergoing the assisted reproductive technologies (ART) treatment.

The bioelectrical impedance (AC electrical resistance value) within the uterine cavity is measured by inserting at least two electrodes into the uterine cavity and bringing them into contact with the endometrium, and applying an AC voltage of a predetermined frequency between the electrodes to pass an AC current. Then, by measuring the current value of the current flowing between each electrode and the endometrium and the potential generated in the endometrium (the potential difference (voltage value) between each electrode), the bioelectrical impedance within the uterine cavity can be detected based on the current value and voltage value.

As the electrodes, platinum electrodes or tungsten electrodes can be suitably used. Further, it is preferable that the electrode has a shape (probe type) that can be inserted into the uterine cavity. Further, regarding the placement of the electrodes, either a two-terminal method in which two electrodes are placed in the uterine cavity or a four-terminal method in which four electrodes are placed may be adopted.

The prediction method of the present embodiment can be performed by comparing the bioelectrical impedance values in the uterine cavity obtained in this manner (these values are collectively referred to as “subject values”) with the bioelectrical impedance values in the uterine cavity obtained from a fertile woman (hereinafter referred to as a “control subject”) (these values are collectively referred to as “control values”).

Here, the term “fertile woman” literally refers to a woman who is capable of becoming pregnant, and preferably refers to a woman who has a history of becoming pregnant.

Incidentally, when sexual intercourse occurs in conjunction with ovulation during the menstrual cycle, the rate of live births per menstrual cycle is thought to be between approximately 10 and 25%. This means that even fertile women (especially those with a history of pregnancy) do not become pregnant every cycle.

In current infertility treatments, under the situation that the assisted reproductive technologies (ART) treatment has become one of the common treatment methods, and it has become possible to clarify whether or not there are any problems in the processes of (i) ovulation and (ii) fertilization and pregnancy, the implantation failure is simply a pathological concept that refers to the situation where pregnancy does not occur despite there being no problems in the above (i) and (ii). This is because there is currently no method for diagnosing implantation failure.

Currently, there are no established criteria for determining whether the uterine implantation capacity is normal. Until now, the uterine implantation capacity has been predicted as the hindsight opinion based on (1) blood progesterone levels, (2) measurement of endometrial thickness under ultrasound, and (3) endometrial biopsy and the resulting presence or absence of pregnancy.

In the infertility patients, the situation is thought to change over the cycle rather than leading to irreversible implantation failure. This means that many patients who are thought to be infertile due to implantation failure can be treated.

Alternatively, it is thought that it is possible to create an endometrial condition suitable for implantation. This is because it is not uncommon for patients to become pregnant spontaneously during the treatment break. Therefore, it is necessary to prospectively evaluate the uterine implantation capacity for each menstrual cycle and provide appropriate treatment.

Therefore, as the result of the above comparison, when the subject value is higher than the corresponding control value, it can be determined that the implantation capacity of the uterus of the subject during that menstrual cycle is insufficient for embryo implantation, i.e., the patient has “insufficient implantation capacity.” On the other hand, when the subject value is the same as or lower than the corresponding control value, the implantation capacity of the uterus of the subject during that menstrual cycle can be judged and determined to be sufficient for embryo implantation, i.e., “good implantation capacity.” For example, when a woman who will undergo the assisted reproductive technologies (ART) treatment is judged and determined to have “insufficient implantation capacity”, the embryo transfer can be canceled and the embryos can be frozen or stored without using previously frozen embryos. After that, the embryo transfer can be postponed until the menstrual cycle when “good implantation capacity” is judged and determined, thereby avoiding unnecessary embryo transfer.

On the other hand, when the woman is judged and determined to have “good implantation capacity,” the implantation capacity of the uterus during that menstrual cycle is deemed appropriate, and the embryo transfer by using a fresh embryo or a thawed and frozen embryo is performed.

In this way, it is possible to provide an efficient infertility treatment, and reduce the financial and physical burden on patients.

Alternatively, the prediction method of the present embodiment can be performed by comparing the subject value with a previously set threshold value.

As the result of the above comparison, when the subject value is higher than the corresponding threshold value, it can be determined that the implantation capacity of the uterus of the subject during that menstrual cycle is insufficient for embryo implantation, i.e., the patient has “insufficient implantation capacity.” On the other hand, when the subject value is the same as or lower than the corresponding threshold value, the implantation capacity of the uterus of the subject during that menstrual cycle can be judged and determined to be sufficient for embryo implantation, i.e., “good implantation capacity.”

### (2) Device for Predicting Uterine Implantation Capacity and Operating Method Thereof

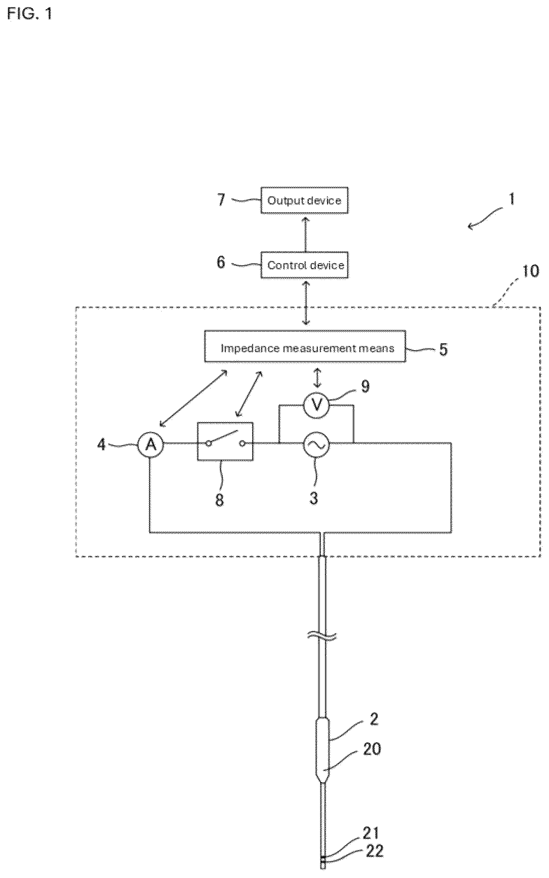

FIG. 1 is a block diagram showing a configuration of the device 1 according to one embodiment of the present invention. The device 1 is composed of a measuring electrode 2, an impedance measuring device 10, a control device 6, and an output device 7.

The measuring electrode 2 is formed in a needle shape (probe type) as a whole so as to be inserted into the uterine cavity of the subject. In the present embodiment, the measuring electrode 2 includes a body portion 20 and electrodes 21 and 22 arranged at the tip of the body portion 20 with a predetermined distance. The tip of the body portion 20 is rounded to prevent it from damaging the inside of the uterus and vagina of the subject. When the electrodes 21 and 22 are inserted into the uterine cavity and brought into contact with the endometrium, a current path is formed between the electrodes 21 and 22 and the endometrium.

When an AC voltage is applied between the electrodes 21 and 22, a current corresponding to the bioelectrical impedance (endometrial impedance) between the electrodes 21 and 22 and the endometrium flows through the above-mentioned current path. Here, an AC voltage of 2.5 to 250 kHz can be used, but the available frequency band is not limited thereto.

The impedance measuring device 10 is provided with a power source 3, an ammeter 4 that measures the current flowing through the above-mentioned current path, a voltmeter 9 that measures the potential difference (voltage value) between the electrodes 21 and 22, and an impedance measurement means 5. The power source 3, the ammeter 4, the voltmeter 5 and the electrodes 21 and 22 are electrically connected to via lead wires.

The power source 3 is a power supply for applying an AC voltage of a predetermined frequency (for example, 2.5 to 250 kHz) between the electrodes 21 and 22, and the ON-OFF control of the application of the voltage to the electrodes 21 and 22 is carried out by a switching element 8. Note that, the ON-OFF operation of the switching element 8 is controlled by the impedance measurement means 5.

The impedance measurement means 5 performs the processes such as controlling the current supply to the electrodes 21 and 22 and controlling the ammeter 4 and voltmeter 9. The impedance measurement means 5 receives an operation signal from the subject, controls the switching element 8 by the ON-OFF control to control the current flow to the electrodes 21 and 22, and calculates the bioelectrical impedance from the voltage value measured by the voltmeter 5 and the current value measured by the ammeter 4. Here, a known method can be used to measure the bioelectrical impedance.

The control device 6 is a semiconductor integrated circuit where the processing which includes the prediction of the implantation capacity of the uterus of the subject is performed by using the bioelectrical impedance value measured by the impedance measuring device 10. The control device 6 can be configured as a microcomputer including a CPU, a ROM, and a RAM. The ROM stores bioelectrical impedance measurement values (control values) of the uterine cavity of the fertile women. The RAM temporarily stores the endometrial impedance of the subject measured by the impedance measuring device 10.

The CPU reads the subject value of the subject stored in the RAM and compares with the control value for fertile women stored in the ROM. Then, when the subject value is higher than the corresponding control value, the implantation capacity of the subject during that menstrual cycle is determined to be insufficient. On the other hand, when the subject value is the same as or lower than the corresponding control value, the implantation capacity of the subject during that menstrual cycle is determined to be good.

The output device 7 is composed of a display device such as a liquid crystal display, and displays the measured value (subject value) of the endometrial impedance measured by the impedance measuring device 10, the assessment result of the uterine implantation capacity of the subject obtained by the control device 6, and the like. Note that the output device 7 is not necessarily limited to a display device, and various types of devices such as a projector, printer, speaker, and the like can be used as long as they are capable of outputting to the outside the measured value (subject value) of the bioelectrical impedance in the uterine cavity measured by the impedance measuring device 10, the measurement results of the uterine implantation capacity of the subject determined by the control device 6, and the like.

According to the device 1 of the present embodiment described above, by bringing the electrodes 21 and 22 into contact with the inside of the uterine cavity of the subject, the potential generated in the endometrium of the subject id detected. By calculating the endometrial impedance of the subject by using the detected potential, it is possible to evaluate the implantation capacity of the uterus in real time. This evaluation allows for prospective evaluation of the uterine implantation capacity for each menstrual cycle and the treatment that results.

### (3) Modification Example of Embodiment 1

### (3-1) Modification Example 1

In the embodiment 1, the uterine implantation capacity of the subject during a certain menstrual cycle is measured by comparing the subject value (measured value of the bioelectrical impedance in the uterine cavity) with the measured value of the bioelectrical impedance in the uterine cavity of a fertile woman (control value). In the modification example 1, the subject value may be compared with each of the previously set threshold value instead of or in addition to the control value.

### (3-2) Modification Example 2

In the embodiment 1, the bioelectrical impedance in the uterine cavity is measured by using the two-terminal method, but the bioelectrical impedance may also be measured by using the four-terminal method.

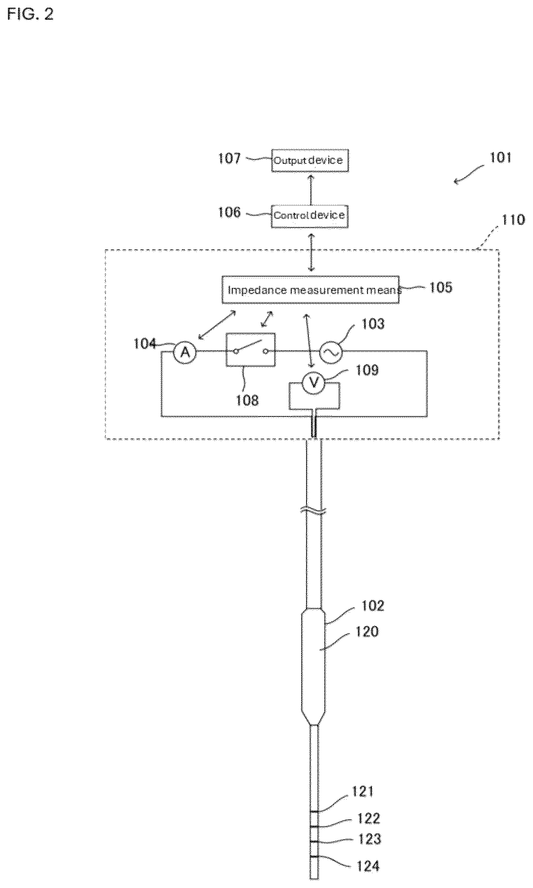

Specifically, the measuring electrode 102 shown in FIG. 2 is formed in a needle shape (probe type) as a whole so as to be inserted into the uterine cavity of the subject. The measuring electrode 102 includes a body portion 120 and electrodes 121 to 124 arranged at the tip of the body portion 120 with a predetermined distance.

The electrodes 121 to 124 are electrically connected to the impedance measuring device 110 via respective lead wires. Specifically, the electrodes are divided into two electrodes for passing current and two electrodes for measuring voltage, and the electrodes 121 and 124 at both ends are electrically connected to an ammeter 104, while the electrodes 122 and 123 are electrically connected to a voltmeter 109.

The impedance measurement means 105 performs the processes such as controlling the current supply to the electrodes 121 to 124 and controlling the ammeter 104 and voltmeter 109. The impedance measurement means 105 receives an operation signal from the subject, controls the switching element 108 by the ON-OFF control to control the current flow to the electrodes 121 and 124, and causes a current to flow through the current path formed between the electrodes 121, 124 and the endometrium. Then, the bioelectrical impedance can be calculated from the current value measured by the ammeter 104 and the voltage value measured by the voltmeter 109.

### 2. Embodiment 2

Embodiment 2 will be described as another embodiment of the present invention. The embodiment 2 relates to estimating the delivery timing or predicting the possibility of preterm delivery.

### (1) Method for Estimating the Delivery Timing

The inventor has found that the difference in the delivery timing from normal times is due to histological changes in the cervix, but the method for estimating the delivery timing in the present embodiment uses the bioelectrical impedance of the vaginal cavity of the subject, that is, vaginal mucosal impedance, as one of the indicators.

Note that, although it has previously been reported that, in the mechanism of the delivery, the compositions of mucus on the cervical and vaginal mucosal epithelium change, the inventors focused on the increase in the sulfate group and the sialic acid group modifications of the glycocalyx, and, from the basis of these material aspects, have reached the understanding that the delivery timing can be estimated by measuring the local bioelectrical impedance.

Specifically, the method for estimating the delivery timing in the present embodiment includes the step of (a) comparing the bioelectrical impedance measurement value at the local position (subject value) in the vaginal cavity measured in the subject with the vaginal mucosal impedance value (control value) previously measured in the fertile woman or the previously set threshold value.

The measurement of the vaginal mucosal impedance is performed by bringing the electrodes into contact with the vaginal mucosal epithelium and measuring the voltage and current between the electrodes under an AC voltage in the predetermined frequency band.

This prediction method includes the step of (b) determining that, when the subject value is lower than the corresponding control value or threshold, the delivery timing in the subject is relatively early (or that the possibility of preterm delivery is relatively high), and determining that, when the subject value is the same as or higher than the corresponding control value or threshold, the delivery timing in the subject is normal or relatively late (or that the possibility of preterm delivery is relatively low).

The inventors believe that changes in the expression of sulfated glycocalyx and/or sialylated glycocalyx (glycoproteins and polysaccharides that coat the cell surfaces) in parts of the placenta and cervical epithelium during the delivery and preterm delivery are reflected in changes in the measured values of the bioelectrical impedance at the local position. Therefore, the above-mentioned method can be used to estimate the delivery timing and predict the possibility of preterm delivery.

### (2) Device for Estimating the Delivery Timing

The device 1 described above can be used as a device for estimating the delivery timing.

However, the control value or threshold to which the subject value is compared may be changed as appropriate.

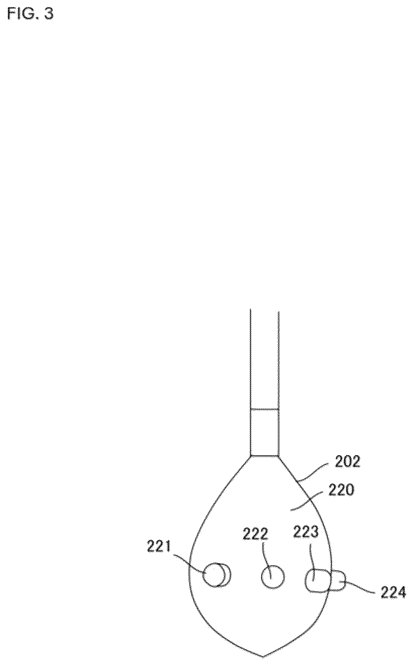

Further, the electrodes are inserted into the vagina. Therefore, as shown in FIG. 3, as the electrodes, a measuring electrode 202 which includes a substantially disk-shaped body portion 220 and electrodes 221 to 224 arranged at predetermined intervals on one side of the body portion 220 is preferably used.

Although the embodiments of the method and the device for predicting the implantation capacity of the uterus and estimating the delivery timing according to the present invention have been described above, the present invention is not limited to these embodiments.

In the embodiment 1, the bioelectrical impedance in the uterine cavity was measured to predict the implantation capacity of the uterus, but instead of or in addition to this, the bioelectrical impedance in the vaginal cavity may be measured and compared with a control value or threshold value.

Further, in the embodiment 2, the bioelectrical impedance of the vaginal cavity is measured to estimate the delivery timing, but instead of or in addition to this, the bioelectrical impedance of the uterine cavity may be measured and compared with a control value or threshold value.

The configuration and effects of the present invention will be specifically explained below by using experimental examples. However, the present invention is not limited to such experimental examples.

All of the following experiments were conducted with the approval of the Ethics Committee of the Osaka University Graduate School of Medicine.

### Experimental Example 1

### Measurement of Bioelectrical Impedance in the Uuterine Cavity in Humans

A device having a sensor-equipped intrauterine catheter (manufactured by TSS Healthcare Co., Ltd.) as an electrode attached to a body composition analyzer (body composition analyzer MLT-550N manufactured by SK Medical Electronics Co., Ltd.) that utilizes the multi-frequency bioelectrical impedance method was manufactured.

The electrodes of this device were inserted into the uterine cavity of the subject, and an AC voltage of 2.5 to 250 kHz and a maximum of 20 mV was generated between the electrodes to measure the bioelectrical impedance. The measurement timings are T1: before ovulation after the end of menstruation (for example, the 9th or 10th day after the start of menstruation), T2: at the timing of ovulation (for example, the day before starting progesterone administration in a hormone replacement cycle), and T3: just before embryo transfer. No adverse events such as bleeding, infection, or abdominal pain were observed. Further, the pregnancy rate after the measurement was 36.2%, this suggests that this measurement does not clearly inhibit pregnancy.

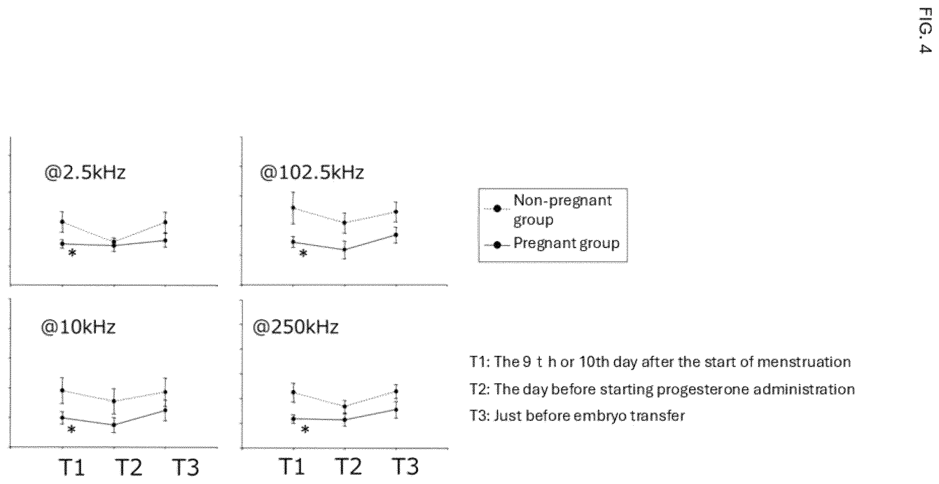

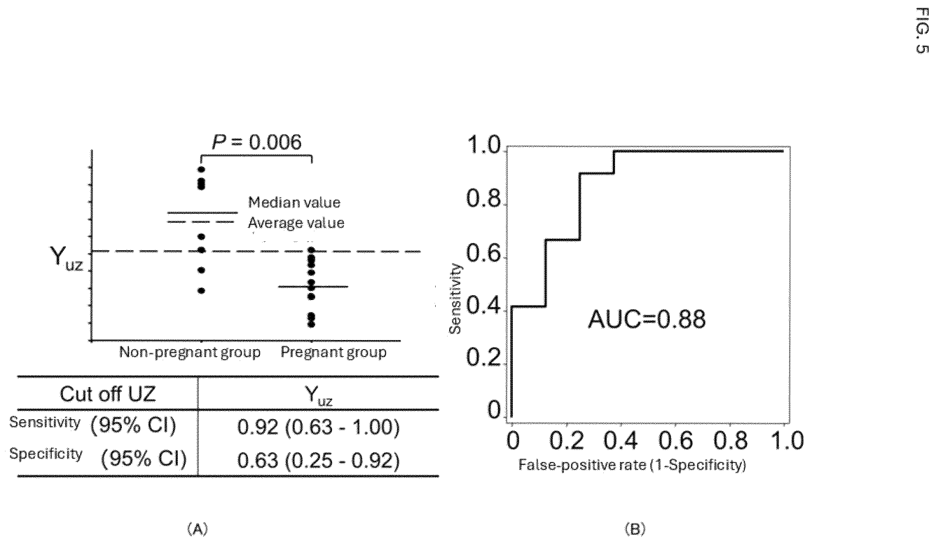

FIG. 4 shows the measurement results for the group that achieved pregnancy and the group that did not achieve pregnancy. Further, FIG. 5 shows the ROC (Receiver Operating Characteristic) analysis of the measurement results in FIG. 4. The AUC (Area Under the ROC Curve) was 0.88, which suggests that the measured values could predict that pregnancy would not occur during this menstrual cycle.

From FIG. 4, a significant difference was observed in the T1 timing under the AC voltages of any frequency. This timing corresponds to the timing when the endometrium begins to thicken. Accordingly, it is clear that by measuring the bioelectrical impedance in the uterine cavity before ovulation after the end of menstruation (for example, on the 9th or 10th day from the start of menstruation), it is possible to predict whether pregnancy will not occur in that menstrual cycle.

This result suggests that, by measuring the bioelectrical impedance value at the local position within the uterine cavity and using this value as an index, it was possible to prospectively predict the implantation capacity of the uterus and reflect this in treatment plans for each menstrual cycle.

### Experimental Example 2

### Measurement of Bioelectrical Impedance at Local Position in the Vaginal Cavity by Using Preterm Delivery Model Mouse

### (1) Preparation of Experimental Animals

### (a) Preterm Delivery Model Mouse

A preterm delivery model mouse was prepared. The first preterm delivery mouse model is a mouse in which preterm delivery is induced by blocking progesterone (progesterone) through administration of Mifepristone (RU-486). Further, the second preterm delivery mouse model is a mouse in which preterm delivery is induced by local infection in the uterus through the administration of lipopolysaccharide (lipopolysaccharide, LPS), a bacterial component.

### (b) Control Mouse

Further, control mouse was prepared for the preterm delivery model mouse in accordance with the description in Non-Patent Document 2.

### (2) Measurement of Vaginal Mucosal Impedance

By using the prepared preterm delivery model mice and control mice, the bioelectrical impedance of the vaginal mucosal epithelium was measured and compared. In this experimental example, a device was manufactured in which a 4-terminal electrode sensor was attached as an electrode to the body composition analyzer (body composition analyzer MLT-550N manufactured by SK Medical Electronics Co., Ltd.) that utilizes the multi-frequency bioelectrical impedance method, and the bioelectrical impedance was measured.

### Experimental Example 2-1

In Experimental Example 2-1, the first preterm delivery model mouse (progesterone-blocked preterm delivery model) administered with Mifepristone was used as the preterm delivery model mouse, and the bioelectrical impedance of the vaginal at the local position was measured 15 hours after the administration.

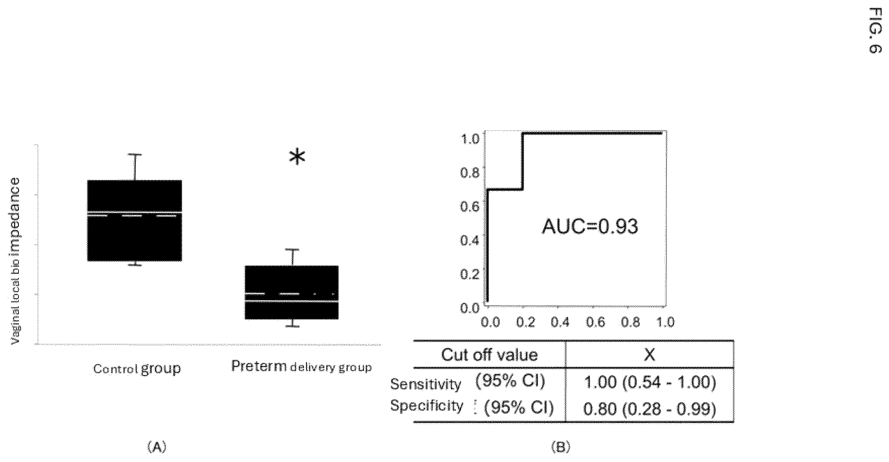

FIG. 6(A) shows a comparison of the bioelectrical impedance measurement values at the local position between two groups: those that ended up in preterm delivery (right) and those that did not end up with preterm delivery (left). FIG. 6(B) shows the results of the ROC analysis of the measurement results in (A). The AUC was 0.93.

### Experimental Example 2-2

In Experimental Example 2-2, the experiment was conducted by using the second preterm delivery model mouse (infectious disease preterm delivery model) administered with LPS as the preterm delivery model mouse. Here, the bioelectrical impedance of the vaginal at the local position was measured 10 hours after the administration of LPS.

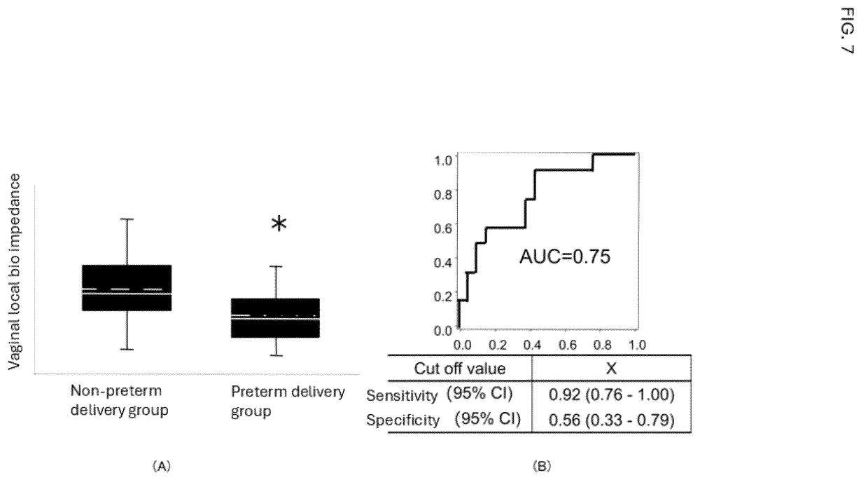

FIG. 7(A) shows a comparison of the bioelectrical impedance measurement values at the local position between two groups: those that ended up in preterm delivery (right) and those that did not end up with preterm delivery (left). FIG. 7(B) shows the results of the ROC analysis of the measurement results in (A). The AUC was 0.75.

From FIG. 6 and FIG. 7, it was confirmed that the measured values of vaginal bioelectrical impedance at the local position in the preterm delivery model mice were significantly lower than those in the control mice. Thus, it was confirmed that the measurement values of the vaginal bioelectrical impedance at the local position show significantly lower values when the preterm delivery occurs. This suggests that the measurement values of the vaginal bioelectrical impedance at the local position can be used as a parameter for estimating the delivery timing (or preterm delivery).

These results suggest that by measuring the vaginal bioelectrical impedance at the local position and using this value as an index, it was possible to estimate the delivery timing (or predict the possibility of preterm delivery) and reflect this in treatment plans.

### Experimental Example 2-3

Next, for Experimental Example 2-3, the first preterm delivery model mouse (progesterone-blocked preterm delivery model) was prepared, to which Mifepristone was administered. Delivery occurred in all first preterm delivery mice 16 to 20 hours after the administration of Mifepristone. Using these first preterm delivery mice, the bioelectrical impedance of the vaginal region 15 hours after the administration of Mifepristone was measured at 33 multiple frequencies in the same manner as described above, and the measured values at five of the frequencies were examined.

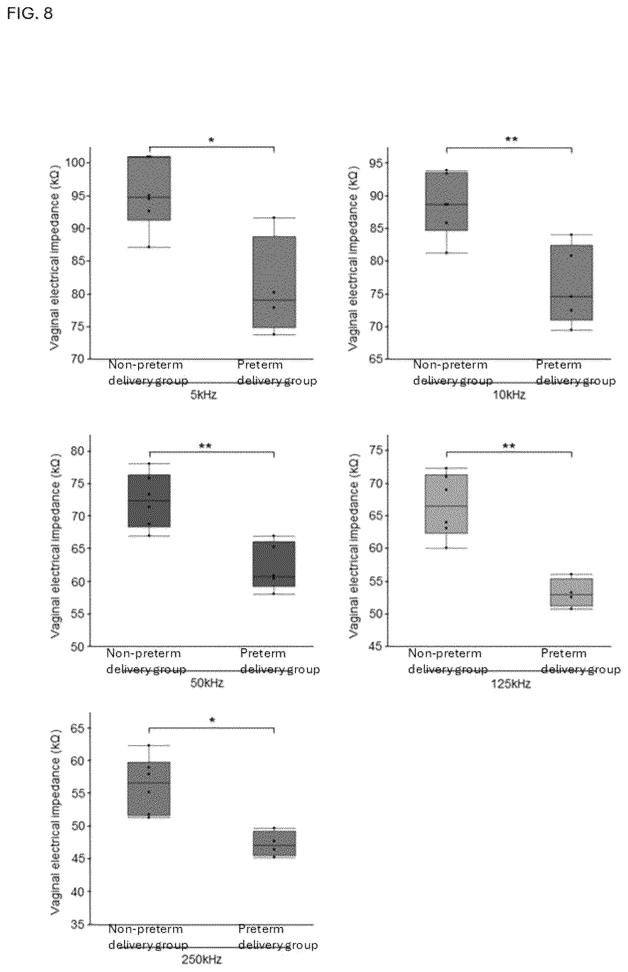

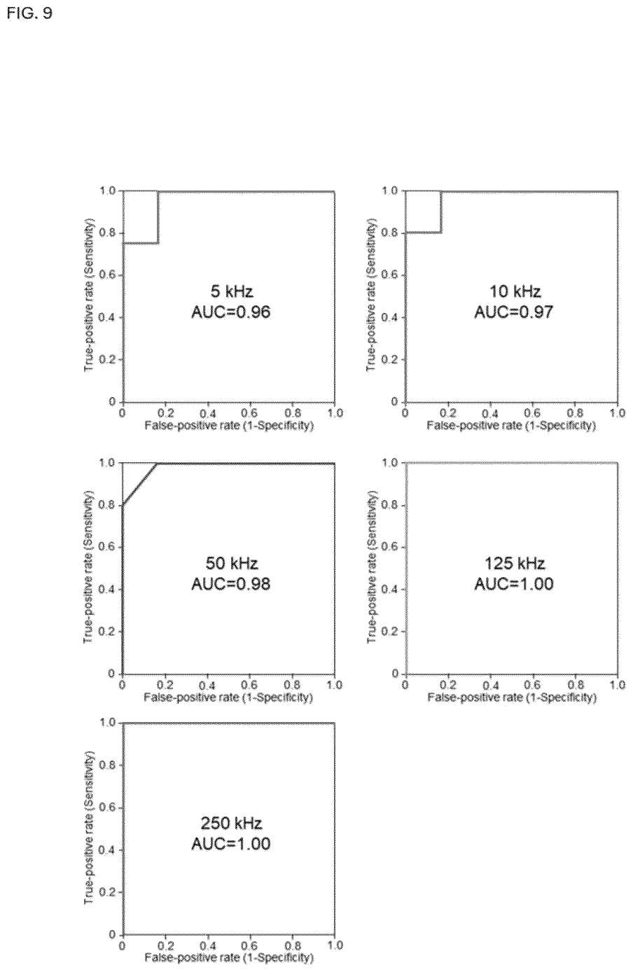

FIG. 8 shows a comparison of the bioelectrical impedance measurement values at the local position taken at frequencies of 5 kHz, 10 kHz, 50 kHz, 125 kHz or 250 kHz between two groups: those that ended up in preterm delivery (right) and those that did not end up with preterm delivery (left). Further, FIG. 9 shows the results of the ROC analysis of the measurement results of FIG. 8. In Experimental Example 2-3, the AUC was also close to 1.00 in relation to continuous frequencies (33 points).

### Experimental Example 2-4

Furthermore, for Experimental Example 2-4, the second preterm delivery model mouse (infectious disease preterm delivery model) was prepared by administering LPS. Using the second preterm mice in which delivery occurred within 24 hours after the administration of 2 μg or 20 μg of LPS, the bioelectrical impedance of the vaginal region 10 hours after LPS administration was measured at 33 multiple frequencies in the same manner as described above, and the measured values at five of the frequencies were examined.

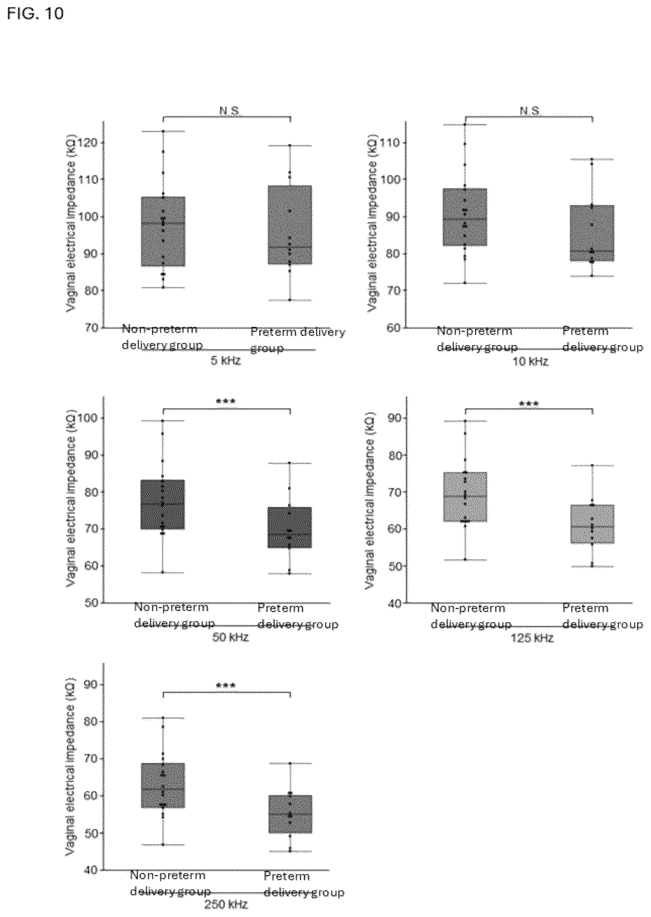

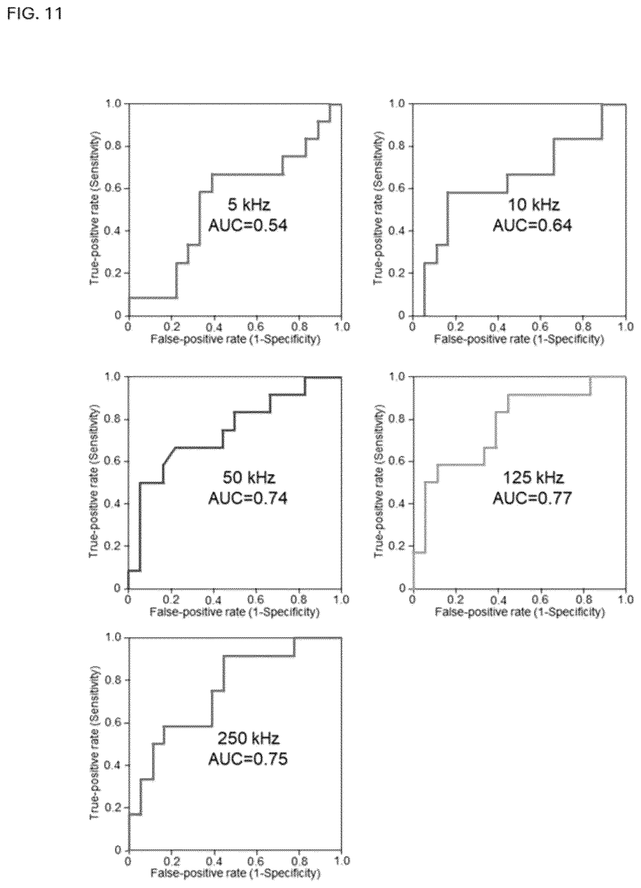

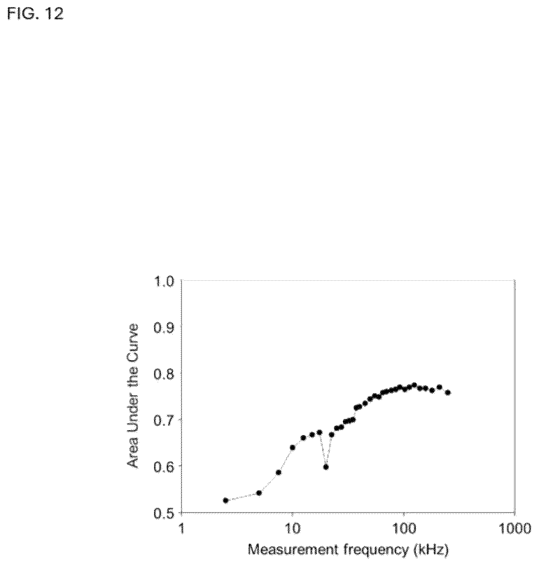

FIG. 10 shows a comparison of the bioelectrical impedance measurement values at the local position taken at frequencies of 5 kHz, 10 kHz, 50 kHz, 125 kHz or 250 kHz between two groups: those that ended up in preterm delivery (right) and those that did not end up with preterm delivery (left). Further, FIG. 11 shows the results of the ROC analysis of the measurement results of FIG. 10. In addition, FIG. 12 shows a graph showing the relationship between successive frequencies and AUC in Experimental Example 2-4.

From FIG. 8 and FIG. 10, it was confirmed that the measured values of vaginal bioelectrical impedance at the local position in the preterm delivery model mice were significantly lower than those in the control mice. Thus, it was confirmed that the measurement values of the vaginal bioelectrical impedance at the local position show significantly lower values when the preterm delivery occurs. This suggests that the measurement values of the vaginal bioelectrical impedance at the local position can be used as a parameter for estimating the delivery timing (or preterm delivery).

Further, from FIG. 9 and FIG. 11, it has been confirmed that, when preterm delivery occurs, the measured values of vaginal local bioelectrical impedance are significantly lower when the second preterm delivery model mouse (infectious disease preterm delivery model) was used than when the first preterm delivery model mouse (progesterone-blocked preterm delivery model) was used.

These results suggest that by measuring the vaginal bioelectrical impedance at the local position by using the second preterm delivery mouse model (infectious disease preterm delivery model) and using this value as an index, it was possible to estimate the delivery timing (or predict the possibility of preterm delivery) and reflect this in treatment plans.

Furthermore, from FIG. 11, it was confirmed that, when the second preterm delivery model mouse (infectious disease preterm delivery model) was used and a high frequency in the range of 50 kHz to 250 kHz was used, the measured value of the vaginal bioelectrical impedance at the local position shows a significantly lower value when preterm delivery occurs, i.e., there was frequency dependency (50 kHz to 250 kHz being preferred).

From FIG. 12, it was confirmed that, when a high frequency of 100 kHz or more or 125 kHz or more was used, the AUC value becomes almost constant to confirm plateaued, and there was a significant frequency dependency (100 kHz to 250 kHz or 125 kHz to 250 kHz being preferable).

Note that, in the above experimental examples, the first group of preterm delivery model mice administered with Mifepristone was analyzed at two doses, 0 μg and 250μg.

On the other hand, in the second group of infectious disease induced preterm delivery model mice, LPS was administered directly into the uterine cavity. As previously reported (Rinaldi et al. (2014) J Immunol 192:2315-25.) the administration of 20 μg resulted in 100% preterm delivery within 24 hours after the administration. In order to investigate whether preterm delivery can be predicted from the measured values of these parameters even under the conditions where preterm delivery was induced in some individuals and not in others at a lower dose of LPS than that which induces 100% preterm delivery, an additional group was administered 2 μg, which was 1/10 the dose, and the mice were divided into two groups: those that ended up in preterm delivery within 24 hours of the administration and those that did not end up with preterm delivery, and preterm delivery was retrospectively predicted from the measured values 10 hours after the administration.

### INDUSTRIAL APPLICABILITY

Infertility is often a temporary imbalance and is said to be a treatable condition. Therefore, it is effective to do prospective diagnosis for each menstrual cycle and treatment adapted to the diagnosis. Thus, by measuring the uterine implantation capacity prospectively in real time, the results can be reflected in infertility treatment.

Specifically, if the measurement results indicate that the uterus has good implantation capacity (the uterus is sufficiently prepared for implantation), embryo transfer is performed by using fresh embryos or frozen embryos. Further, if it is determined that the uterus has insufficient implantation capacity (the uterus is not ready for implantation), embryo transfer is not performed for that cycle and the embryos is cryopreserved, and embryo transfer is not performed for that cycle.

Accordingly, by performing embryo transfer in a cycle in which the uterine implantation capacity is judged to be suitable, it is possible to provide a highly efficient infertility treatment. That is, since the number of unnecessary embryo transfers decrease, the number of times as to ovarian hyperstimulation and egg retrieval, which have been performed repeatedly until now, can also be reduced.

Further, by measuring the bioelectrical impedance of the vaginal area after implantation and using this value as an index, it is possible to estimate the delivery timing (or predicting the possibility of preterm delivery). That is, the existing predictive parameters for preterm delivery have a high negative predictive rate. By using this parameter in combination with known parameters, it is possible to select cases that require aggressive medical intervention, and provide the necessary treatment to those who need it.

### EXPLANATION OF SYMBOLS

1,101 Device
2,102 Electrode
6,106 Control device
7,107 Output device
10,110 Impedance measuring device

1. A device for estimating the delivery timing of a subject, comprising:
an electrode that is brought into contact with the mucosal epithelium at a position in either the uterine cavity or the vaginal cavity of the subject;
a measurement means that measures the bioelectrical impedance generated between the electrode and the mucosal epithelium; and
an estimating means that estimates the delivery timing of the subject by using the measured value of the bioelectrical impedance as an index.

2. The device according to claim 1, wherein the estimating means includes:
a comparison means for comparing the measured value with at least one of a predetermined threshold value and a control value which is a value of local bioelectrical impedance at a position in the uterine cavity or vaginal cavity previously measured in a fertile woman; and
a determination means for determining that the delivery timing of the subject is early when the measurement value is lower than the corresponding threshold value or control value.

3. The device according to claim 1, which further comprises an output means for outputting the estimated delivery timing.

4. A method, which comprises the steps of:
bring an electrode into contact with the mucosal epithelium at a position in either the uterine cavity or the vaginal cavity of a subject;
measuring the local bioelectrical impedance at the position; and
estimating the delivery timing of the subject by using the measured value of the bioelectrical impedance as an index.

5. A device for predicting the implantation capacity of the uterus of a subject, which comprises:
an electrode that is inserted up to a position which is either in the uterine cavity or intravaginal cavity of the subject;
a measurement means for measuring the local bioelectrical impedance at the position; and
a predicting means for predicting the implantation capacity by using the measured local bioelectrical impedance as an index.

6. The device according to claim 5, wherein the predicting means includes:
a comparison means for comparing the measured value with at least one of a predetermined threshold value and a control value which is a value of local bioelectrical impedance at a position in the uterine cavity or vaginal cavity previously measured in a fertile woman; and
a determination means for determining that the implantation capacity in the corresponding menstrual cycle is insufficient when the measured value is higher than the corresponding threshold value or control value, and for determining that the implantation capacity in the corresponding menstrual cycle is good when the measured value is the same as or lower than the corresponding threshold value or control value.

7. The device according to claim 5, which further comprises an output means for outputting the predicted implantation capacity.

8. A method which comprises the steps of:
inserting an electrode up to a position which is either in the uterine cavity or intravaginal cavity of a subject;
measuring the local bioelectrical impedance at the position; and
predicting the implantation capacity by using the measured local bioelectrical impedance as an index.
